(19) United States
(12) Patent Application Publication
(10) Pub. No.: US 2024/0115534 A1
(43) Pub. Date: Apr. 11, 2024

(54) COMPOSITION FOR MUSCLE ENHANCEMENT AND USE OF SAME

(71) Applicant: J-OIL MILLS, INC., Tokyo (JP)

(72) Inventors: Masayoshi SAKAINO, Tokyo (JP); Toshiro SATO, Tokyo (JP); Shigeo TAKEUCHI, Tokyo (JP)

(21) Appl. No.: 18/275,913

(22) PCT Filed: Feb. 7, 2022
(86) PCT No.: PCT/JP2022/004719

§ 371 Date: Aug. 4, 2023

(30) Foreign Application Priority Data
Feb. 19, 2021 (JP) 2021-025441
Aug. 6, 2021 (JP) 2021-129674

(51) Int. Cl. A61K 31/20; A61K 31/23; A61P 21/00

(52) U.S. Cl. CPC A61K 31/20; A61K 31/23; A61P 21/00

(57) ABSTRACT

There is provided a novel ingredient that exhibits an exceptional effect for enhancing muscle using a fatty acid.

A composition for muscle enhancement, characterized in that the composition contains, as an active component, at least one selected from the group consisting of C13, C14, or C15 fatty acids and fatty acid esters that include these fatty acids. The fatty acid is preferably at least one selected from tridecanoic acid, tetradecanoic acid, or pentadecanoic acid. The composition for muscle enhancement is suitably used in the form of, inter alia, an oil and fat composition, a food or beverage, a supplement, or an animal feed.

### TECHNICAL FIELD

The present invention relates to a composition for muscle enhancement and a use of the same.

### BACKGROUND ART

Maintaining muscle function is important for improving quality of life and prolonging healthy life. This is because muscle function is not merely for exercise and activities that are necessary to lead daily life, but also has aspects in which such exercise and activities for leading daily life relieve mental stress. Additionally, in the aging society of recent years, there are also an increasing number of cases where an aging-associated decline in muscle function results in further hindrance to other bodily functions and, oftentimes, development of clinically notable diseases.

There has been a focus of late on health-promoting-function aspects of fatty acids (Non-patent Document 1). However, there have been no reports focusing on the use of fatty acids for muscle enhancement.

### RELATED ART DOCUMENTS

### [Non-Patent Documents]

[Non-patent Document 1] KAYA, Kunimitsu. Live longer and healthier! Introduction to functional fatty acids: Effects on Alzheimer's disease, cancer, diabetes, and memory recovery. Shokabo Co., Ltd., February 2017 issue

### DISCLOSURE OF THE INVENTION

### Problems the Invention is Intended to Solve

It is an object of the present invention to provide a novel ingredient that exhibits an exceptional effect for enhancing muscle using a fatty acid.

### Means for Solving the Problems

The inventors conducted thorough investigations in order to achieve the aforementioned object and finally perfected the present invention. Specifically, the present invention is as described below.
[1] A composition for muscle enhancement, characterized in that the composition contains, as an active component, at least one selected from the group consisting of C13, C14, or C15 fatty acids and fatty acid esters that include these fatty acids.
[2] The composition for muscle enhancement according to [1] above, wherein the fatty acid is at least one selected from tridecanoic acid, tetradecanoic acid, or pentadecanoic acid.
[3] The composition for muscle enhancement according to [1] or [2] above, wherein the fatty acid ester is a glycerin fatty acid ester.
[4] The composition for muscle enhancement according to any of [1] to [3] above, containing 0.01 mass % or more and 100 mass % or less of at least one selected from the group consisting of the fatty acids and the fatty acid esters.
[5] The composition for muscle enhancement according to any of [1] to [4] above, having an effect for enlarging muscle fiber.
[6] An oil and fat composition containing the composition for muscle enhancement according to any of [1] to [5] above.
[7] A food or beverage containing the composition for muscle enhancement according to any of [1] to [5] above.
[8] A supplement containing the composition for muscle enhancement according to any of [1] to [5] above.
[9] An animal feed containing the composition for muscle enhancement according to any of [1] to [5] above.

### Effect of the Invention

According to the present invention, it is possible to provide a novel ingredient that exhibits an exceptional effect for enhancing muscle using a fatty acid.

### MODE FOR CARRYING OUT THE INVENTION

The fatty acid used in the present invention is preferably a C13, C14, or C15 fatty acid, but there is no particular limitation as to, inter alia, the source from which the fatty acid is derived. For example, the fatty acid may be derived from a natural substance or may be synthetic. Additionally, the fatty acid is preferably usable as a C13, C14, or C15 fatty acid upon breaking down within a living body; for example, a fatty acid ester that includes the fatty acid may be used. In order to provide the fatty acid in the form of a food or beverage, a supplement, an animal feed, or the like, it is preferable for the fatty acid to be prepared from a natural substance. For ease of explanation, there are cases where the C13, C14, or C15 fatty acid or the fatty acid ester containing the aforementioned fatty acid used in the present invention is referred to simply as “fatty acid (C13-15)” or “fatty acid (C13-15) ester,” or as “fatty acid (C13-15) and/or fatty acid ester thereof.”

Specific examples of the fatty acid (C13-15) include tridecanoic acid (C13:0), tetradecanoic acid (C14:0), and pentadecanoic acid (C15:0). Such fatty acids are not provided by way of limitation; the fatty acid (C13-15) may be an unsaturated fatty acid having a double bond in part of the fatty acid alkyl chain or part of the fatty acid alkyl chain may be branched. Examples of the fatty acid (C13-15) ester include glycerin fatty acid esters, sucrose fatty acid esters, organic-acid fatty acid esters, fatty acid alcohol esters, sphingolipids, sorbitan fatty acid esters, polypropylene fatty acid esters, and sterol esters that contain the fatty acid (C13-15). Among these, glycerin fatty acid esters are preferred. Glycerin fatty acid esters have glycerin as a constituent component. Specific examples of glycerin fatty acid esters include triacylglycerols, diacylglycerols, monoacylglycerols, glycerophospholipids, and glyceroglycolipids. One type of fatty acid (C13-15) and/or fatty acid ester thereof may be used alone, or a plurality thereof may be used in combination. When enantiomers or diastereomers are present, one type may be used alone as a monomolecular species thereof, or a plurality of types of multiple molecular species of these isomer pairs may be used in combination.

The method for obtaining the fatty acid (C13-15) from a natural substance is not limited to what is described below. However, for example, oils and fats such as coconut oil, palm kernel oil, and milk fat include numerous triglyceride-containing lipids including the fatty acid (C14) as a constituent fatty acid. Additionally, JP-A 2016-89025 clarifies that large amounts of triglycerides including the fatty acid (C13) and the fatty acid (C15) as constituent fatty acids are produced in algae of the genus Aurantiochytrium, which are known as algae that produce large amounts of oil and fat. Thus, using such edible oils and fats or algae that produce large amounts of oil and fat as base materials makes it possible to efficiently obtain the fatty acid (C13-15). Typically, triglyceride-containing lipids including the fatty acid (C13) and the fatty acid (C15) as constituent fatty acids are obtained by, e.g., adding an organic solvent such as n-hexane, chloroform, diethyl ether, methanol, or ethanol, a mixed organic solvent obtained from these organic solvents, or the like to a dried form or another form of a natural substance such as algae and then extracting the triglyceride-containing lipids. Polar lipids may be removed from the resultant triglyceride-containing lipids through adsorption using silica gel, acid clay, activated clay, an ion-exchange resin, or the like to obtain triglycerides. Moreover, impurities may be removed from the triglyceride-containing lipids through ordinary steps for refining vegetable oils and fats, i.e., a degumming step, a deacidification step, a decoloration step, or a deodorization step, to obtain refined triglyceride-containing lipids. Furthermore, the resultant triglycerides, triglyceride-containing lipids, or refined triglyceride-containing lipids can be subjected to a sodium hydroxide treatment or a hydrolysis treatment carried out using a lipase or the like, making it possible for fatty acids to be isolated from the ester structure of the glycerin. The isolated fatty acids may furthermore be separated and refined through molecular distillation, urea addition, column chromatography, or another method. The resultant fatty acids, triglycerides, triglyceride-containing lipids, or refined triglyceride-containing lipids may furthermore be used as raw materials in transesterification or ester synthesis of triglycerides.

When an ingredient containing the fatty acid (C13-15) and/or fatty acid ester thereof having a natural substance as a base material is formulated and used, it is, for example, preferable to use an ingredient in which the content value (purity) for the fatty acid (C13-15) and/or fatty acid ester thereof is raised to 1 mass % or greater, more preferable to use an ingredient in which said content value is raised to 2 mass % or greater, even more preferable to use an ingredient in which said content value is raised to 5 mass % or greater, and particularly preferable to use an ingredient in which said content value is raised to 10 mass % or greater. Alternatively, it is, for example, preferable to use an ingredient in which the total content value for other fatty acids other than the fatty acid (C13-15) is 99 mass % or less, more preferable to use an ingredient in which said content value is 95 mass % or less, even more preferable to use an ingredient in which said content value is 90 mass % or less, and particularly preferable to use an ingredient in which said content value is 80 mass % or less.

The fatty acid (C13-15) and/or fatty acid ester thereof can be quantified using methods that are well known to persons skilled in the art. For example, the fatty acid (C13-15) and/or fatty acid ester thereof can be quantified by, inter alia, carrying out analysis using gas chromatography, liquid chromatography, or the like as established in Standard Methods for the Analysis of Fats, Oils and Related Materials by the Japan Oil Chemists' Society, and adapting to the concentration found in analysis of a standard sample established in a discretionary manner. In the present specification, quantitative identification of the fatty acid (C13-15) and/or fatty acid ester thereof can be handled using the total amount of one or a plurality of types of fatty acids (C13-15) and/or esters thereof that can be sensed.

The composition for muscle enhancement according to the present invention can be in the form of an oil and fat composition containing the fatty acid (C13-15) and/or fatty acid ester thereof but is not limited to this form. Specifically, for example, an edible oil and fat, an excipient, an adjuvant, an emulsifier, a pH adjuster, and the like can be blended in a discretionary manner as necessary to produce an oil and fat composition in the form of a liquid, powder, paste, or other discretionary form using a well-known technique. For example, the aforementioned ingredients may be formulated into a liquid oil and fat, margarine, fat spread, shortening, powdered oil and fat, or the like that is mainly composed of oil and fat components, or may be formulated into a dissolved, powdered, gelled, granular, or other form in which few oils and fats are blended; these forms can be employed in a discretionary manner. Additionally, for example, in the case of powderization, corn syrup or another adjuvant can be used, and an emulsifier may furthermore be added to formulate an emulsion raw material, which may then be powderized. Examples of means for powderization include spray drying, freeze-drying, and the like.

Examples of the edible oil and fat include: rapeseed oil (including high-oleic-acid varieties), soybean oil (including high-oleic-acid varieties), palm oil, palm kernel oil, corn oil, olive oil, sesame oil, safflower oil, sunflower oil (including high-oleic-acid varieties), cottonseed oil, rice bran oil, peanut oil, coconut oil, grapeseed oil, cacao butter, and other vegetable oils and fats; algae oil, beef tallow, pig lard, chicken fat, milk fat, and other animal oils and fats; and medium-chain fatty acid triglycerides. Additional examples include fractionated oils (such as medium-melting-point fractionated oils of palm oil, soft fractionated oils of palm oil, and hard fractionated oils of palm oil), transesterified oils, hydrogenated oils, and other processed oils and fats obtained from these edible oils and fats. One edible oil and fat may be used alone, or two or more may be blended together.

Additives that are normally added to edible oil and fat compositions may be blended into the oil and fat composition as appropriate. Examples of additives include antioxidants, defoamers, emulsifiers, fragrances, flavor-imparting agents, pigments, and biologically active substances. Specific examples include ascorbic acid fatty acid esters, lignan, coenzyme Q, γ-oryzanol, tocopherol, and silicone.

The content value for the fatty acid (C13-15) and/or fatty acid ester thereof to be incorporated into the oil and fat composition is not particularly limited. For example, this content value may be within the range of 0.01 mass % or greater and 100 mass % or less, the range of 0.1 mass % or greater and 100 mass % or less, the range of 1 mass % or greater and 80 mass % or less, the range of 1 mass % or greater and 50 mass % or less, the range of 1 mass % or greater and 30 mass % or less, the range of 1 mass % or greater and 10 mass % or less, or the range of 2 mass % or greater and 10 mass % or less. Alternatively, the content value may be within the range of 10 mass % or greater and 30 mass % or less. The ingredient for providing the fatty acid (C13-15) and/or fatty acid ester thereof itself may constitute the composition for muscle enhancement.

In the composition for muscle enhancement according to the present invention, the fatty acid (C13-15) and/or fatty acid ester thereof is used for muscle enhancement.

The wording “for muscle enhancement” can specifically refer to increasing or maintaining muscle mass, or increasing or maintaining muscle strength, via an effect for enlarging muscle fiber. Specifically, as indicated in the test examples that shall be described later, the fatty acid (C13-15) has an effect for enlarging the myotube area and/or the maximum transverse diameter of myotube cells. Myotube cells are elongated coenocytes formed through fusion of myoblasts, and muscle fiber is produced when myotube cells form fiber bundles. Therefore, the enlargement of the myotube area and/or the maximum transverse diameter of the myotube cells caused by the action of the fatty acid (C13-15) is associated with enlargement of muscle fiber and, by extension, leads to an increase in or maintenance of muscle mass or to an increase in or maintenance of muscle strength. Thus, for example, the fatty acid (C13-15) is also useful for improving or preventing conditions such as sarcopenia or frailty, in which a decrease in muscle mass due to aging or illness results in a decline in overall muscle strength and body function. The fatty acid (C13-15) is additionally useful for applications such as: “promoting an increase in or maintenance of muscle mass and, in combination with exercise, increasing or maintaining muscle strength or suppressing any decrease in muscle strength”; “relieving temporary muscle fatigue associated with exercise”; “improving or maintaining exercise capabilities”; and “increasing energy metabolism in association with an increase in muscle mass, thus improving physical constitution or improving/eliminating metabolic syndrome.” Additionally, when imparted to farm animals or aquatic animals, the fatty acid (C13-15) is also useful for producing meat that is richer in proteins.

The composition for muscle enhancement according to the present invention can be suitably used in, e.g., children in a growth stage or able-bodied persons in their prime or middle/old age. More specifically, the composition for muscle enhancement can be more suitably used in able-bodied persons aged 40 or older. The composition for muscle enhancement is not limited to use in humans, but rather can also be applied to: livestock such as cows, pigs, chickens, sheep, and horses; pets such as dogs and cats; and other non-human animals.

The composition for muscle enhancement according to the present invention is preferably used such that the fatty acid (C13-15) and/or fatty acid ester thereof is administered to a subject, but the present invention is not particularly limited to this mode of use. For example, the fatty acid (C13-15) and/or fatty acid ester thereof is preferably incorporated into a prescribed dosage form, and the dosage form is preferably administered in a desired mode as appropriate, such as being orally administered, applied to the skin, administered through absorption, or intramuscularly injected. The content value for the fatty acid (C13-15) and/or fatty acid ester thereof in the dosage form is preferably adjusted as appropriate from the standpoint of ensuring a desired dosage amount and is not particularly limited. For example, this content value may be within the range of 0.1 mass % or greater and 100 mass % or less, the range of 1 mass % or greater and 100 mass % or less, the range of 1 mass % or greater and 80 mass % or less, the range of 1 mass % or greater and 50 mass % or less, the range of 2 mass % or greater and 30 mass % or less, the range of 10 mass % or greater and 50 mass % or less, the range of 10 mass % or greater and 30 mass % or less, or the range of 30 mass % or greater and 50 mass % or less.

It is known that enzymes for isolating free fatty acids from esters are ordinarily present in the body, examples of these enzymes including lingual lipase present in the oral cavity, gastric lipase present in the stomach, and pancreatic lipase secreted within pancreatic juice. Furthermore, triglycerides and other esters included in the blood are broken down into free fatty acids and glycerin by lipoprotein lipase present on the surfaces of cells such as muscle cells. Moreover, phospholipases include classes of lipases for isolating free fatty acids from glycerophospholipids, which are esters, through hydrolysis. Additionally, it is known that various free-fatty-acid intake receptors such as GPR40, GPR41, GPR43, and GPR120, which are G protein-coupled receptors (GPRs), are present on cell surfaces. Thus, within the body of a subject to whom the fatty acid ester of the fatty acid (C13-15) is administered, the fatty acid (C13-15) is isolated from the fatty acid ester as appropriate in a timely manner, and the effects of the fatty acid can be exhibited within the body.

The amount of the fatty acid (C13-15) and/or fatty acid ester thereof to be administered when a human orally ingests the composition for muscle enhancement according to the present invention can be determined, as appropriate, according to the age or physical condition of the person to whom the composition is administered, an administration continuance period, the frequency of administration, or the like. In terms of an amount per day for an adult, the ordinary administration amount that is exemplified may be, e.g., within the range of 5 mg or more and 5000 mg or less, the range of 50 mg or more and 3000 mg or less, the range of 80 mg or more and 2000 mg or less, or the range of 80 mg or more and 1000 mg or less.

The composition for muscle enhancement according to the present invention may be formulated into, e.g., an oil and fat composition, a food or beverage, a supplement, or an animal feed, together with suitable additives, preparational ingredients, and the like.

Typical examples of the form of an oil and fat composition include margarine, butter, fat spread, and shortening.

Typical examples of the form of a food or beverage include confectionery (such as potato chips and other snack foods, cookies or cakes and other baked confectionery, Japanese-style confectionery, chocolate, and candy), desserts (such as pudding, gelatin desserts, and ice cream), breads (such as sweet buns, stuffed breads, croissants, and Danishes), noodles (such as ramen, udon, and pastas), rice-based foods (such as rice balls, rice gruel, and fried rice), cereal foods (such as corn flakes and oatmeal), dairy products (such as cheese and yogurt), processed meat products (such as ham and sausage), processed seafood products (such as kamaboko and fish sausage), condiments (such as mayonnaise, Worcestershire sauce, and dressings), soups (such as miso soup and vegetable soups), processed foods and beverages (such as stewed foods, fried foods, grilled foods, and curry), pre-mixed flours (such as okonomiyaki flour, deep-frying flour, and confectionery mix flours), solid roux (such as curry roux), beverages (such as: beer and other alcohol; sports drinks, lactic acid beverages, or vegetable juice and other soft drinks; black tea and other teas; and coffee), elderly-oriented foods and beverages (such as liquid foods), health foods, health-promoting foods, foods for specified health uses, foods labeled with functions, functional nutritional foods, nutritional supplement foods, and supplements.

Typical examples of the form of a supplement include tablets, pills, capsules, powdered medicines, granules, liquid medicines, syrups, jelly formulations, and candy medications.

Typical examples of the form of an animal feed include: feeds for livestock such as cows, pigs, chickens, sheep, and horses; feeds for small-sized animals such as rabbits, rats, and mice; feeds for marine animals such as eels, sea bream, young yellowtail, and shrimp; and feeds for pet animals such as dogs, cats, small birds, squirrels, and turtles.

### Examples

The present invention is described more specifically below by way of test examples, but the scope of the present invention is in no way limited by these test examples.

The materials and methods used in the present test examples are as follows.

(1. Cells)

Cells of mouse myoblast strain C2C12 (ATCC catalog no. CRL-1772)

(2. Culture Medium)

Proliferation culture medium: DMEM (high glucose) to which 10% FBS and 1% PS are added

Differentiation culture medium: DMEM (high glucose) to which 2% HS and 1% PS are added

(FBS: fetal bovine serum (made by Sigma-Aldrich Co., LLC); PS: penicillin/streptomycin mixed solution (made by Nacalai Tesque Inc.); HS: horse serum; DMEM (high glucose): Dulbecco's Modified Eagle Medium (high glucose) (made by Sigma-Aldrich Co., LLC))

(3. Substances Under Test)

The fatty acids indicated below were added to the culture medium so as to reach a concentration of 0.05 mM, 0.2 mM, or 0.5 mM. As a positive control, insulin-like growth factor (IGF-1) (made by Sigma-Aldrich Co., LLC), which is a factor for promoting formation of myotube cells, was added to the culture medium so as to reach a concentration of 10 ng/mL.

(1) Medium-chain fatty acid; mixture of octanoic acid (C8:0) (99.2% purity, made by Sigma-Aldrich Co., LLC) and decanoic acid (C10:0) (99.1% purity, made by Sigma-Aldrich Co., LLC) in ratio of 75 mass %:25 mass %
(2) Undecanoic acid (C11:0) (99.9% purity, made by Sigma-Aldrich Co., LLC)
(3) Tridecanoic acid (C13:0) (99.2% purity, made by Sigma-Aldrich Co., LLC)
(4) Tetradecanoic acid (C14:0) (100% purity, made by Sigma-Aldrich Co., LLC)
(5) Pentadecanoic acid (C15:0) (98.8% purity, made by Sigma-Aldrich Co., LLC)
(6) Hexadecanoic acid (C16:0) (99% purity, made by Sigma-Aldrich Co., LLC)
(7) Heptadecanoic acid (C17:0) (99% purity, made by Sigma-Aldrich Co., LLC)

### 4. Formulation of Samples

The fatty acids were dissolved in dimethyl sulfoxide (DMSO) (made by Nacalai Tesque Inc.) to formulate solutions having a concentration of 0.05 M, 0.2 M, 0.5 M, or 1 M, and the solutions were added in 1/1000 amounts to the culture medium to reach a target final concentration of 0.05 mM, 0.2 mM, 0.5 mM, or 1 mM. The IGF-1 serving as a positive control was formulated to a concentration of 10 μg/mL using 0.1 N HCl, and the resultant solution was added in a 1/1000 amount to the culture medium to reach a target final concentration of 10 ng/mL.

### 5. Cell Culturing and Treatment with Substance Under Test

The C2C12 cells were recovered in a T75 flask using the proliferation culture medium and cultured using a CO2 incubator (37° C., 5% CO2, moist; same applies below). The culture medium was replaced at intervals of one to two days. The cells were peeled away (3 to 5 minutes) using 0.25% trypsin-EDTA (made by Nacalai Tesque Inc.) (1 mL) at the point in time when 80% confluence was reached, and 5 mL of culture medium was added, after which the components were centrifuged (180×g, 5 minutes, room temperature). A supernatant was removed, and cell pellets were suspended in fresh culture medium and sown in a 96-well black plate at a rate of 10,000 cells/0.1 mL/well. The next day, the culture medium was replaced with the differentiation culture medium, and culturing was carried out for two days to induce differentiation into myotube cells. The culture medium was then replaced with differentiation culture medium (100 μL) to which the substance under test was added, and culturing was carried out for a further two days.

### 6. Staining Reagent

Primary antibody reagent: anti-myosin heavy chain (MHC) antibodies (made by eBioscience Inc., 14-6503-82)

Secondary antibody reagent: fluorescent-labeled anti-mouse IgG2b antibodies (made by Thermo Fisher Scientific Inc., A21147)

Nucleus-staining reagent: Hoechst 33342 (made by Thermo Fisher Scientific Inc., H3570)

### 7. Immunostaining

After treatment with the substance under test, the wells were refilled with 100 μL of a fixing solution (4% PFA: paraformaldehyde (made by Nacalai Tesque Inc.)), incubated for 15 minutes at 4° C., and then washed three times using Dulbecco's Phosphate-Buffered Saline (DPBS) (made by Thermo Fisher Scientific Inc.). 100 μL of a blocking/membrane permeation liquid solution (0.3% TritonX/3% BSA/DPBS) was added, and the components were incubated for 30 minutes at room temperature. The aforementioned solution replaced with a primary antibody solution (primary antibody reagent (diluted by a factor of 300)/3% BSA/DPBS) and then incubated overnight at 4° C. The primary antibody solution was removed, and the wells were washed three times using a blocking solution (3% BSA/DPBS) before being refilled with a secondary antibody solution (secondary antibody reagent (diluted by a factor of 500)/0.1% nucleus-staining reagent/3% BSA/DPBS) and incubated for two hours at room temperature. Finally, the wells were washed three times using DPBS and refilled with new DPBS before imaging analysis was carried out.

### 8. Imaging Analysis

An automatic cell image system (“Operetta CLS,” made by PerkinElmer Inc.) was used in the imaging analysis of the immunostained cells. Nine fields of view of central sections of the wells were imaged using a 10× objective lens, and then fluorescent regions marked by the anti-MHC antibodies and cell nucleus regions marked by the nucleus-staining reagent were detected using image analysis software (Harmony 4.6) included with the system. Because myotube cells are fused cells, fused cells including three or more cell nuclei were assessed to be myotube cells, and each of the myotube area of the myotube cells within the fields of view ((myotube area)/(field of view)) and the per-cell maximum transverse diameter of the myotube cells ((maximum transverse diameter)/(myotube cell)) were measured using n=5. The transverse diameter was derived by drawing an inscribed circle in each cell and measuring the diameter of the circle.

### 9. Statistical Analysis

The statistical significance of a group treated with IGF-1 and that of a group treated with the substance under test relative to a group not treated with the substance under test were examined using Dunnett's test. The significance level was 5% for both.

### 10. Results

The average values and standard deviation of the resultant (myotube area)/(field of view) and (maximum transverse diameter)/(myotube cell) were expressed as absolute values (%), where 100% is the average value for the control group that was not treated with the substance under test. The results shown in tables 1-4 indicate results achieved in mutually distinct data acquisition periods. In cases where test results achieved using the same substance under test and final concentration are shown, the values obtained in the respective data acquisition periods are shown.

TABLE 1

| | Treatment with | | | | | | | |
|---|---|---|---|---|---|---|---|---|
| | substance under test | | | | | Maximum transverse | | |
| | | Final | Myotube area/field of view (%) | | | diameter/myotube cell (%) | | |
| | Substance | concentration | Average | Standard | Statistical | Average | Standard | Statistical |
| | under test | (mM) | value | deviation | significance | value | deviation | significance |
| Comparative | None added | — | 100.0 | 1.7 | | 100.0 | 1.5 | |
| example 1-1 | | | | | | | | |
| Comparative | IGF-1 | 10 ng/mL | 114.1 | 1.7 | * | 101.8 | 1.4 | |
| example 1-2 | | | | | | | | |
| Comparative | Medium-chain | 0.5 | 101.5 | 1.3 | | 99.0 | 1.9 | |
| example 1-3 | fatty acid | | | | | | | |
| Comparative | Undecanoic | 0.5 | 103.4 | 1.9 | | 103.1 | 0.7 | |
| example 1-4 | acid (C11:0) | | | | | | | |
| Example 1-1 | Tridecanoic | 0.5 | 98.1 | 3.9 | | 124.1 | 8.5 | * |
| | acid (C13:0) | | | | | | | |
| Example 1-2 | Pentadecanoic | 0.5 | 116.0 | 2.8 | * | 132.2 | 2.8 | * |
| | acid (C15:0) | | | | | | | |
| Comparative | Heptadecanoic | 0.5 | 82.8 | 1.6 | * | 98.4 | 3.4 | |
| example 1-5 | acid (C17:0) | | | | | | | |

TABLE 2

| | Treatment with | | | | | | | |
|---|---|---|---|---|---|---|---|---|
| | substance under test | | | | | Maximum transverse | | |
| | | Final | Myotube area/field of view (%) | | | diameter/myotube cell (%) | | |
| | Substance | concentration | Average | Standard | Statistical | Average | Standard | Statistical |
| | under test | (mM) | value | deviation | significance | value | deviation | significance |
| Comparative | None added | — | 100.0 | 1.5 | | 100.0 | 2.2 | |
| example 2-1 | | | | | | | | |
| Comparative | IGF-1 | 10 ng/mL | 126.8 | 1.8 | * | 99.1 | 1.6 | |
| example 2-2 | | | | | | | | |
| Example 2-1 | Pentadecanoic | 0.05 | 108.0 | 1.5 | * | 103.0 | 2.2 | |
| Example 2-2 | acid (C15:0) | 0.2 | 119.3 | 0.9 | * | 109.3 | 1.7 | * |
| Example 2-3 | | 0.5 | 118.7 | 0.5 | * | 111.4 | 1.7 | * |

TABLE 3

| | Treatment with | | | | | | | |
|---|---|---|---|---|---|---|---|---|
| | substance under test | | | | | Maximum transverse | | |
| | | Final | Myotube area/field of view (%) | | | diameter/myotube cell (%) | | |
| | Substance | concentration | Average | Standard | Statistical | Average | Standard | Statistical |
| | under test | (mM) | value | deviation | significance | value | deviation | significance |
| Comparative | None added | — | 100.0 | 1.3 | | 100.0 | 1.4 | |
| example 3-1 | | | | | | | | |
| Comparative | IGF-1 | 10 ng/mL | 120.5 | 2.4 | * | 100.4 | 1.1 | |
| example 3-2 | | | | | | | | |
| Example 3-1 | Tetradecanoic | 0.5 | 114.7 | 1.4 | * | 108.6 | 1.6 | * |
| Example 3-2 | acid (C14:0) | 1 | 107.3 | 3.4 | | 124.4 | 3.7 | * |

TABLE 4

| | Treatment with | | | | | | | |
|---|---|---|---|---|---|---|---|---|
| | substance under test | | | | | Maximum transverse | | |
| | | Final | Myotube area/field of view (%) | | | diameter/myotube cell (%) | | |
| | Substance | concentration | Average | Standard | Statistical | Average | Standard | Statistical |
| | under test | (mM) | value | deviation | significance | value | deviation | significance |
| Comparative | None added | — | 100.0 | 1.7 | | 100.0 | 1.2 | |
| example 4-1 | | | | | | | | |
| Comparative | IGF-1 | 10 ng/mL | 132.8 | 3.0 | * | 104.8 | 1.0 | |
| example 4-2 | | | | | | | | |
| Comparative | Hexadecanoic | 0.05 | 91.4 | 3.7 | | 98.7 | 1.5 | |
| example 4-3 | acid (C16:0) | | | | | | | |
| Comparative | | 0.5 | 80.9 | 4.4 | * | 104.9 | 3.3 | |
| example 4-4 | | | | | | | | |

As shown in table 1, when pentadecanoic acid (C15:0), which is a C15 fatty acid, was added at a concentration of 0.5 mM, the area of the myotube cells within the fields of view and the per-cell maximum transverse diameter of the myotube cells both significantly increased (example 1-2) over comparative example 1-1, which served as a control. Also, when tridecanoic acid (C13:0), which is a C13 fatty acid, was added at a concentration of 0.5 mM, the per-cell maximum transverse diameter of the myotube cells within the fields of view significantly increased (example 1-1) over comparative example 1-1, which served as a control. However, when heptadecanoic acid (C17:0), which is a C17 fatty acid, was added at a concentration of 0.5 mM, the area of the myotube cells within the fields of view significantly decreased (comparative example 1-5) below comparative example 1-1, which served as a control. Moreover, even if a medium-chain fatty acid, which was a mixture of C8 and C10 fatty acids, or undecanoic acid (C11:0), which is a C11 fatty acid, was added at a concentration of 0.5 mM, no effect on the myotube cells was observed (comparative examples 1-3 and 1-4).

Additionally, as shown in table 2, when the C15 fatty acid pentadecanoic acid (C15:0) was added at a concentration of 0.2 mM or 0.5 mM, the area of the myotube cells within the fields of view and the per-cell maximum transverse diameter of the myotube cells both significantly increased (examples 2-2 and 2-3) over comparative example 2-1, which served as a control. Also, even when the C15 fatty acid pentadecanoic acid (C15:0) was added at a concentration of 0.05 mM, the per-cell maximum transverse diameter of the myotube cells within the fields of view significantly increased (example 2-1) over comparative example 2-1, which served as a control.

Additionally, as shown in table 3, when tetradecanoic acid (C14:0), which is a C14 fatty acid, was added at a concentration of 0.5 mM, the area of the myotube cells within the fields of view and the per-cell maximum transverse diameter of the myotube cells both significantly increased (example 3-1) over comparative example 3-1, which served as a control. Also, when the C14 fatty acid tetradecanoic acid (C14:0) was added at a concentration of 1.0 mM, the per-cell maximum transverse diameter of the myotube cells within the fields of view significantly increased over comparative example 3-1, which served as a control, and a trend was observed in which this effect increased dependently upon the concentration of the C14 fatty acid tetradecanoic acid (example 3-2).

However, as shown in table 4, even when hexadecanoic acid (C16:0), which is a C16 fatty acid, was added in a concentration of 0.05 mM or 0.5 mM, no effect on the per-cell maximum transverse diameter of the myotube cells within the fields of view was observed (comparative examples 4-3 and 4-4). Also, when the C16 fatty acid hexadecanoic acid (C16:0) was added in a concentration of 0.5 mM, the area of the myotube cells within the fields of view significantly decreased (comparative example 4-4) below comparative example 4-1, which served as a control.

1. A method for muscle enhancement, comprising administering a composition for muscle enhancement to a subject, the composition containing, as an active component, at least one selected from the group consisting of C13, C14, or C15 fatty acids and fatty acid esters that include these fatty acids.

2. The method for muscle enhancement according to claim 1, wherein the fatty acid is at least one selected from tridecanoic acid, tetradecanoic acid, or pentadecanoic acid.

3. The method for muscle enhancement according to claim 1, wherein the fatty acid ester is a glycerin fatty acid ester.

4. The method for muscle enhancement according to claim 1, the composition containing 0.01 mass % or more and 100 mass % or less of at least one selected from the group consisting of the fatty acids and the fatty acid esters.

5. The method for muscle enhancement according to claim 1, the composition having an effect for enlarging muscle fiber.

6. The method for muscle enhancement according to claim 1, the composition being in a form of an oil and fat composition.

7. The method for muscle enhancement according to claim 1, the composition being in a form of a food or beverage.

8. The method for muscle enhancement according to claim 1, the composition being in a form of a supplement.

9. The method for muscle enhancement according to claim 1, the composition being in a form of an animal feed.
